(19) United States
(12) Patent Application Publication
(10) Pub. No.: US 2020/0338372 A1
(43) Pub. Date: Oct. 29, 2020

(54) OXYGEN GENERATOR

(71) Applicant: O2-Matic Products Private Limited, Bangalore (IN)

(72) Inventor: John Paul Thambusami Joy Sachidanadam, BANGALORE (IN)

(21) Appl. No.: 16/508,415

(22) Filed: Jul. 11, 2019

(30) Foreign Application Priority Data
Apr. 26, 2019 (IN) 201941016754

(51) Int. Cl. A62B 21/00; A62B 7/08

(52) U.S. Cl. CPC A62B 21/00; A61M 16/10; A62B 7/08

(57) ABSTRACT

An oxygen generator is provided. The oxygen generator includes a generator case comprising at least two hollow chambers, at least two cartridges housed within the corresponding at least two hollow chambers. Each of the at least two cartridges include a water chamber, a chemical chamber, a holding means mechanically coupled to an interior top portion of the at least two cartridges, a triggering means mechanically held in place by the holding means. The triggering means include a top seal configured to unlock a bottom seal upon twisting a trigger connector, a trigger valve, a hollow seal tube located along a central axis of the corresponding at least two cartridges, the bottom seal configured to allow a pre-defined amount of water to enter the chemical chamber via a filter. The hollow seal tube is configured to allow flow of oxygen generated in the chemical chamber.

### CROSS-REFERENCE TO RELATED APPLICATION

This application claims the benefit of complete patent application having Patent Application No. 201941016754 filed on Apr. 26, 2019 in India.

### FIELD OF INVENTION

Embodiments of a present invention relate to an oxygen generator, and more particularly to an oxygen generator.

### BACKGROUND

Oxygen is a type of a colourless and an odourless gas which majorly supports life on earth. In certain situation, oxygen is stored in tanks and provided to people having certain breathing issues or undergoing operation so as to maintain a desired level of oxygen in breathing air. In certain other cases, tanks storing oxygen under pre-defined pressure referred to as oxygen cylinders is also used by people in other situations such as travel in space shuttle, scuba diving, mountaineering and the like for maintaining required supply of oxygen. However, such oxygen cylinders are usually composed of metals and are heavy in weight and big in size which creates a problem for transportation or porting of such cylinders. Bulky oxygen cylinders make it very difficult to carry them around. Also, refilling of oxygen into the cylinder is a lengthy and expensive process. Thereby oxygen generators are being used to enable easy porting of the oxygen.

In one such approach, an oxygen generator is used to generate and store the oxygen upon combining a number of chemicals together in a vessel at a required pressure. Consequently, the oxygen generator generates the oxygen at any instant of time. However, rate of flow of generation of oxygen by the oxygen generator cannot be maintained and also duration for which the oxygen generator can generate oxygen is limited or short. Such limitation may not produce a required quantity of oxygen for a desired duration of time. Also, a user need to manually mix number of chemicals in order to generate the oxygen. Such human interventions can lead to inaccuracy in ratio of the number of chemicals, thereby making the oxygen generator less efficient and less accurate.

Hence, there is a need for an improved oxygen generator to address the aforementioned issues.

### BRIEF DESCRIPTION

In accordance with one embodiment of the disclosure, an oxygen generator is provided. The oxygen generator includes a generator case. The generator case includes at least two hollow chambers. The oxygen generator also includes at least two cartridges housed within the corresponding at least two hollow chambers. Each of the at least two cartridges include a water chamber configured to store a pre-defined amount of water. Each of the At least two cartridges also include a chemical chamber mechanically coupled to the water chamber. The chemical chamber is configured to store one or more chemicals in a pre-defined ratio. The water chamber is placed above the chemical chamber and is separated by a disc and is pneumatically sealed. Each of the At least two cartridges also include a holding means mechanically coupled to an interior top portion of the corresponding at least two cartridges. Each of the At least two cartridges also include a triggering means mechanically held in place by the holding means. The triggering means include a top seal configured to unlock a bottom seal upon twisting a trigger connector to a pre-defined angle. The triggering means also include a trigger valve operatively coupled to the top seal. The trigger valve is configured to enable a process of generation of oxygen. The triggering means also includes a hollow seal tube located along a central axis of the corresponding at least two cartridges. The triggering means also includes the bottom seal operatively coupled at a bottom of the hollow seal tube. The bottom seal is configured to allow the pre-defined amount of water to enter the chemical chamber via a filter. The filter is fixed to the disc within the chemical chamber. The hollow seal tube is configured to allow flow of oxygen generated from chemical chamber to the water chamber. The hollow seal tube is also configured to allow flow of oxygen generated in the chemical chamber upon reaction of the pre-defined amount of water with the one or more chemicals to exit from the water chamber. The at least two cartridges are placed in the corresponding at least two hollow chambers of the generator case.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a schematic cross-sectional view of an oxygen generator in accordance with an embodiment of the present disclosure;

FIG. 2 is a schematic representation of an embodiment of an external view of the oxygen generator of FIG. 1 in accordance with an embodiment of the present disclosure;

FIG. 3 is a schematic representation of another embodiment of an external view of the oxygen generator of FIG. 1 in accordance with an embodiment of the present disclosure;

FIG. 4 is a schematic representation of an embodiment representing an internal view of the oxygen generator of FIG. 1 in accordance with an embodiment of the present disclosure;

FIG. 5 is a schematic representation of an oxygen generator with a humidifier means in accordance with an embodiment of the present disclosure; and

FIG. 6 is a schematic representation of an embodiment representing housing of the oxygen generator with the humidifier means of FIG. 5 in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to an oxygen generator. The oxygen generator includes a generator case. The generator case includes at least two hollow chambers. The oxygen generator also includes at least two cartridges housed within the corresponding at least two hollow chambers. Each of the at least two cartridges include a water chamber configured to store a pre-defined amount of water. Each of the At least two cartridges also include a chemical chamber mechanically coupled to the water chamber. The chemical chamber is configured to store one or more chemicals in a pre-defined ratio. The water chamber is placed above the chemical chamber and is separated by a disc and is pneumatically sealed. Each of the At least two cartridges also include a holding means mechanically coupled to an interior top portion of the corresponding at least two cartridges. Each of the At least two cartridges also include a triggering means mechanically held in place by the holding means. The triggering means include a top seal configured to unlock a bottom seal upon twisting a trigger connector to a pre-defined angle. The triggering means also include a trigger valve operatively coupled to the top seal. The trigger valve is configured to enable a process of generation of oxygen. The triggering means also includes a hollow seal tube located along a central axis of the corresponding at least two cartridges. The triggering means also includes the bottom seal operatively coupled at a bottom of the hollow seal tube. The bottom seal is configured to allow the pre-defined amount of water to enter the chemical chamber via a filter. The filter is fixed to the disc within the chemical chamber. The hollow seal tube is configured to allow flow of oxygen generated from chemical chamber to the water chamber. The hollow seal tube is also configured to allow flow of oxygen generated in the chemical chamber upon reaction of the pre-defined amount of water with the one or more chemicals to exit from the water chamber. The at least two cartridges are placed in the corresponding at least two hollow chambers of the generator case.

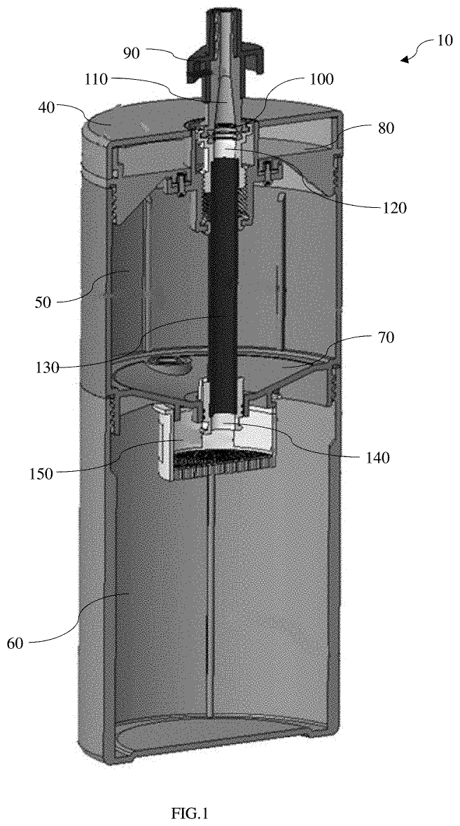

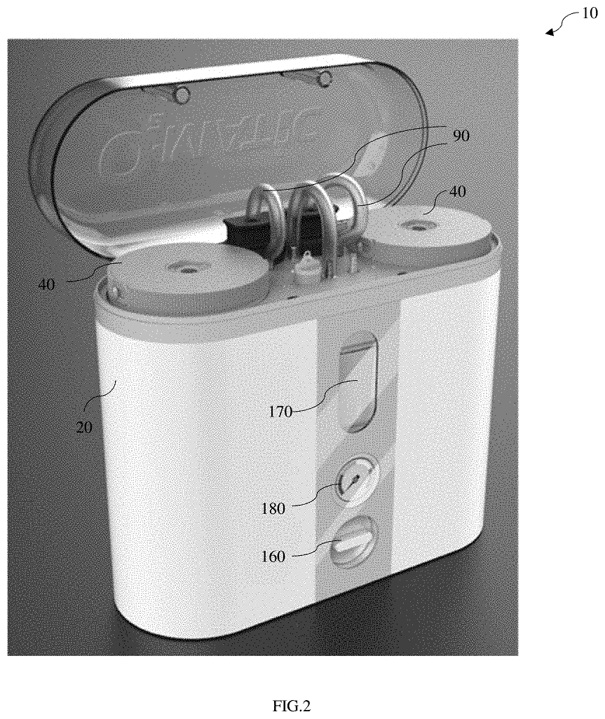

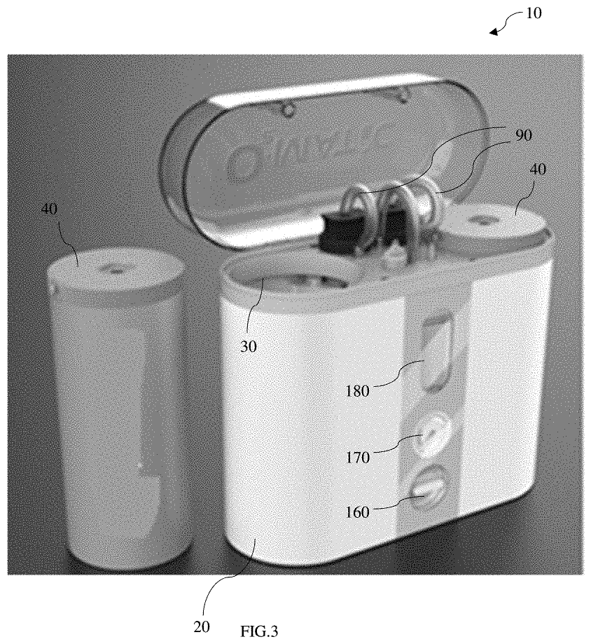

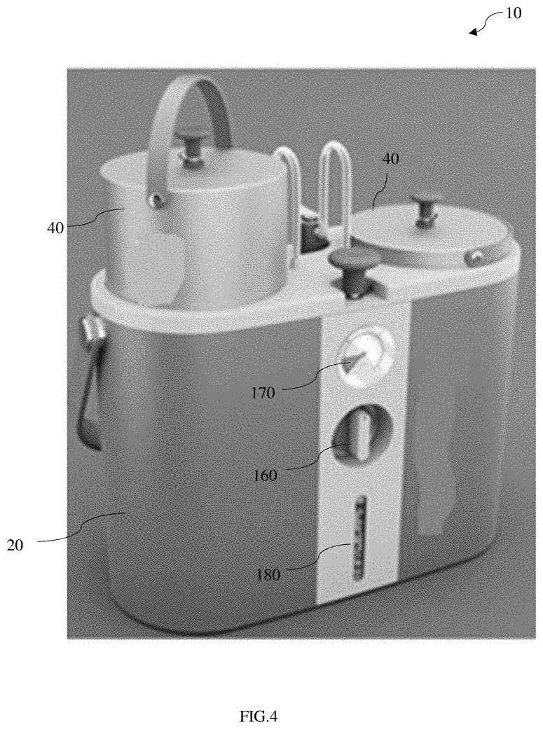

FIG. 1 is a schematic cross-sectional view of an oxygen generator 10 in accordance with an embodiment of the present disclosure. As used herein, the term “oxygen generator” is defined as a medical device that generates oxygen as a result of a reaction of one or more elements. The oxygen generator 10 includes a generator case 20 (as shown in FIG. 2, FIG. 3 and FIG. 4) comprising at least two hollow chambers 30. In one embodiment, each of the at least two hollow chambers 30 may correspond to a hollow cylindrical chamber. The at least two hollow cylindrical chambers may be separated by a solid material organised in a pre-defined shape.

The oxygen generator 10 also includes at least two cartridges 40 housed within the corresponding at least two hollow chambers 30. As used herein, the term “cartridge” is defined as a hollow container for holding material used for an insertion into a mechanism. In one exemplary embodiment, each of the at least cartridges 40 are coupled with a handle which is configured to operate the at least two cartridges 40. In such embodiment, operation of the at least two cartridges 40 may include loading and unloading the at least two cartridges 40 into the corresponding at least two hollow chambers 30. Furthermore, each of the at least two cartridges 40 include a water chamber 50. The water chamber 50 is configured to store a pre-defined amount of water.

Each of the at least two cartridges 40 also include a chemical chamber 60 mechanically coupled to the water chamber 50. The chemical chamber 60 is configured to store one or more chemicals in a pre-defined ratio. In one embodiment, the one or more chemicals includes at least one of sodium percarbonate, potassium superoxide, peroxide species (hydrogen peroxide), urea-hydrogen peroxide and percarbamide peroxide. In one exemplary embodiment, the one or more chemicals may be in a form of one of a granular powder, pellets, powder in a pouch and candle stick.

Furthermore, the water chamber 50 is placed above the chemical chamber 60 and is separated by a disc 70 and is pneumatically sealed. Each of the at least two cartridges 40 also include a holding means 80 mechanically coupled to an interior top portion of the corresponding at least two cartridges 40.

Each of the at least two cartridges 40 also include a triggering means 90 mechanically held in place by the holding means 80. As used herein, the term “triggering means” is defined as a means used to trigger or cause a particular reaction based on a particular action. The triggering means 90 includes a top seal 100 configured to unlock a bottom seal 140 upon twisting a trigger connector 110 to a pre-defined angle. In one embodiment, the top seal 100 may be twisted manually by a user to the pre-defined angle. In such embodiment, the pre-defined angle may be 90 degrees. In one embodiment, the trigger connector 110 may be placed on a top surface of the generator case 20. The trigger connector 110 may be configured to keep the connection of the at least two cartridges 40 in place during the generation of oxygen.

Also, the triggering means 90 includes a trigger valve 120 operatively coupled to the top seal 100. The trigger valve 120 is configured to enable a process of generation of oxygen. More specifically, the generation of the oxygen will be initiated upon enabling the one or more chemicals to react with the pre-defined amount of water. In order to enable the reaction, the top seal 100 is first triggered and correspondingly the trigger valve 120 is operated to enable the process of generation of oxygen.

The triggering means 90 also includes a hollow seal tube 130 located along a central axis of the corresponding at least two cartridges 40. As used herein, the term “central axis” is defined as an imaginary straight line passing vertically about a centre of an object. The triggering means 90 also includes the bottom seal 140 operatively coupled at a bottom of the hollow seal tube 130. The bottom seal 140 is configured to allow the pre-defined amount of water to enter the chemical chamber 60 via a filter 150. The filter 150 is fixed to the disc within the chemical chamber 60. More specifically, the pre-defined amount of water stored in the water chamber 50 is made to pass to the chemical chamber 60 on twisting the top seal 100.

Furthermore, the hollow seal tube 130 is configured to allow flow of oxygen generated from chemical chamber 60 to the water chamber 50. More specifically, as the bottom seal 140 is made open, the pre-defined amount of water reacts with the one or more chemicals in the chemical chamber 60 and generate oxygen. Consequently, the generated oxygen is passed through the hollow seal tube 130. In addition, the hollow seal tube 130 is also configured to allow flow of oxygen generated in the chemical chamber 60 upon reaction of the pre-defined amount of water with the one or more chemicals to exit from the water chamber 50. In one embodiment, the flow of oxygen in the hollow seal tube 130 is uni-directional and may be controlled by the bottom seal 140.

In one exemplary embodiment, the chemical chamber 60 of each of the at least two cartridges 40 may include a mechanical stirrer (not shown in FIG. 1). The mechanical stirrer may be configured to keep the one or more chemicals in the chemical chamber 60 in constant mixing.

In another exemplary embodiment, the oxygen generator 10 may include an oxygen flow control knob 160 operatively coupled to each of the at least two cartridges 40 via the corresponding trigger connector 110. The oxygen flow control knob 160 may be configured to control flow ratio of oxygen generated by each of the at least two cartridges 40. In one specific embodiment, the trigger connector 110 may be configured to prevent disconnection of trigger connector 110 from the corresponding at least two cartridges 40 during the generation of oxygen.

In one specific embodiment, the oxygen generator 10 may further include a humidifier 180 operatively coupled to the oxygen flow control knob 160. The humidifier 180 may be configured to impart humidity to the generated oxygen. As used herein, the term “humidity” is defined as amount of water vapour present in air.

Furthermore, in an exemplary embodiment, the oxygen generator 10 may further include an oxygen level indicator 170 operatively coupled to each of the at least two cartridges 40. The oxygen level indicator 170 may be configured to display oxygen level in each of the at least two cartridges 40. More specifically, the oxygen level indicator 170 may indicate the level of oxygen generated by each of the at least two cartridges 40. In another exemplary embodiment, the oxygen generator 10 may further include an oxygen pressure regulator (not shown in FIG. 1) operatively coupled to each of the at least two cartridges 40. The oxygen pressure regulator may be configured to enable a uniform flow of oxygen. In yet another exemplary embodiment, the oxygen generator 10 may further include a cooling mechanism (not shown in FIG. 1) operatively coupled to each of the at least two cartridges 40. The cooling mechanism may be configured to dissipate heat from each of the at least two cartridges 40. In yet another exemplary embodiment, the oxygen generator 10 may further include at least one storage tank (not shown in FIG. 1) operatively coupled to at least one of the at least two cartridges 40. The at least one storage tank may be configured to enable a uniform flow of oxygen generated in the chemical chamber upon storing the generated oxygen

In one specific embodiment, the oxygen generator 10 may be composed of polymer. In one exemplary embodiment, the at least two cartridges 40 may be sealed within the corresponding at least two hollow chambers 30 by placing and rotating the corresponding at least two cartridges 40 in a clockwise direction. In such embodiment, the at least two cartridges 40 may be sealed within the corresponding at least two hollow chambers 30 by using a bayonet mount mechanism. In such another embodiment, the at least two cartridges 40 may be removed from the corresponding at least two hollow chambers 30 by rotating the at least two cartridges 40 in an anti-clockwise direction.

In operation, the triggering connector 110 of the triggering means 90 of a first cartridge of the at least one cartridge 40 is manually twisted to 90 degrees, consequently the top seal 100 and hence the trigger valve 120 of the triggering means 90 of the first cartridge is opened to allow the flow of pre-defined amount of water from the water chamber 50 to enter the chemical chamber 60 via the filter 150 which is controlled by the bottom seal 140. Further, as the pre-defined amount of water is mixed with the one or more chemicals, oxygen is released as a product. Further, the oxygen generated in the chemical chamber 60 is transferred to thee outlet via the hollow seal tube 130 passing through the water chamber 50. The oxygen generated in the chemical chamber 60 is then transferred to the humidifier 180 to impart humidity to the generated oxygen. Also, the level of oxygen left in the first cartridge is indicated by the oxygen level indicator 170. Furthermore, as the oxygen generated in the first cartridge gets ceased, the triggering connector 110 of the triggering means 90 of a second cartridge of the at least one cartridge 40 is manually twisted to 90 degrees, and the process of generation of oxygen is repeated in the second cartridge as in the first cartridge.

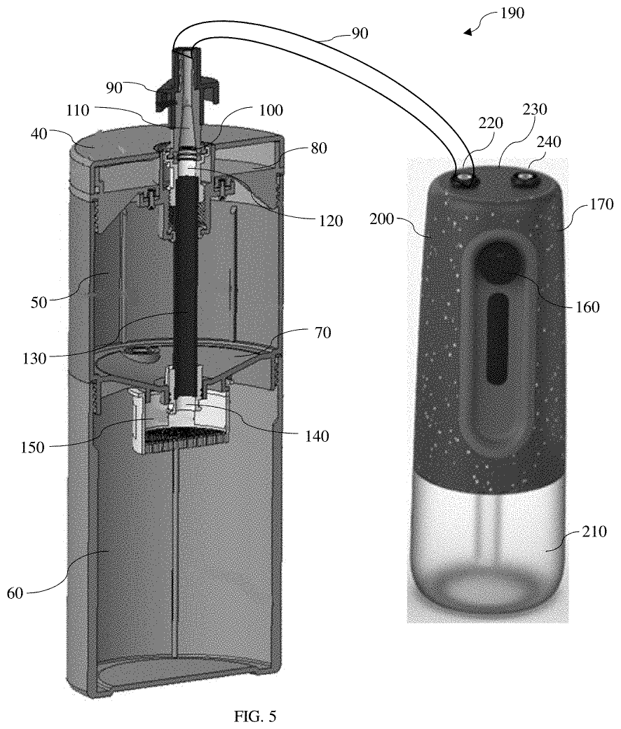

FIG. 5 is a schematic representation of an oxygen generator 190 with a humidifier means 200 in accordance with an embodiment of the present disclosure. The oxygen generator 190 includes at least one cartridge 40. The at least one cartridge 40 includes a water chamber 50. The water chamber 50 is configured to store a pre-defined amount of water. The at least one cartridge 40 also includes a chemical chamber 60 mechanically coupled to the water chamber 50. The chemical chamber 60 is configured to store one or more chemicals in a pre-defined ratio. The water chamber 50 is placed above the chemical chamber 60 and is separated by a disc 70. In addition, the at least one cartridge 40 is pneumatically sealed.

Furthermore, the at least one cartridge 40 also includes a holding means 80 mechanically coupled to an interior top portion of the corresponding at least one cartridge 40. The at least one cartridge 40 also includes a triggering means 90 mechanically held in place by the holding means 80. The triggering means 90 includes a top seal 100. The top seal 100 is configured to unlock a bottom seal 140 upon twisting a trigger connector 110 to a pre-defined angle. The triggering means 90 also includes a trigger valve 120 operatively coupled to the top seal 100. The trigger valve 120 is configured to enable a process of generation of oxygen. The triggering means 90 also includes a hollow seal tube 130 located along a central axis of the corresponding at least one cartridge 40. Also, the bottom seal 140 is operatively coupled at a bottom of the hollow seal tube 130. The bottom seal 140 is operatively coupled at a bottom of the hollow seal tube 130. The bottom seal 140 is configured to allow the pre-defined amount of water to enter the chemical chamber 60 via a filter 150. The filter 150 is fixed to the disc 70 within the chemical chamber 60.

Moreover, the hollow seal tube 130 is configured to allow flow of oxygen generated from chemical chamber 60 to the water chamber 50. The hollow seal tube 130 is also configured to allow flow of oxygen generated in the chemical chamber 60 upon reaction of the pre-defined amount of water with the one or more chemicals to exit from the water chamber 50.

The oxygen generator 190 also includes the humidifier means 200 operatively coupled to the at least one cartridge 40 through the triggering means 90. The humidifier means 200 includes a container 210 configured to store water. The humidifier means 200 also includes an oxygen flow control knob 160 operatively coupled to the container 210. The oxygen flow control knob 160 is configured to control flow ratio of oxygen generated by the at least one cartridge 40. The humidifier means 200 also includes an inlet valve 220 operatively coupled to a top surface 230 of the humidifier means 200. The inlet valve 220 is configured to allow the flow of generated oxygen from the at least one cartridge 40 to the container 210. The humidifier means 200 also includes an outlet valve 240 operatively coupled to the top surface 230 of the humidifier means 200. The outlet valve 240 is configured to allow the flow of humidified oxygen generated in the container 210.

Furthermore, the at least one cartridge 40 comprising the water chamber 50, the chemical chamber 60, the holding means 80, and the triggering means 90 are substantially similar to at least two cartridges 40 comprising a water chamber 50, a chemical chamber 50, a holding means 80 and a triggering means 90 of FIG. 1. Also, the humidifier means 200 comprising the oxygen flow knob 160 is substantially similar to a humidifier 180 coupled to an oxygen flow control knob 160 as described in FIG. 1.

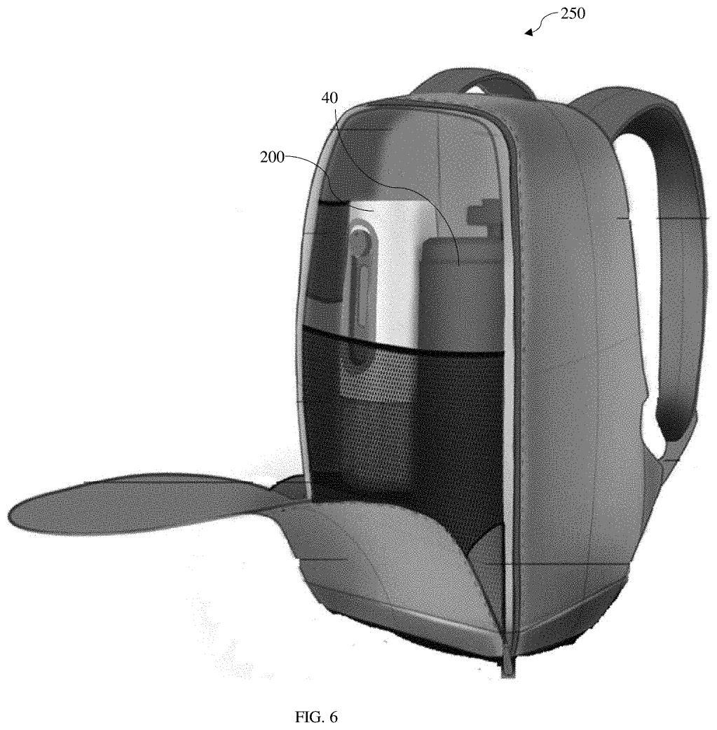

In one exemplary embodiment, the oxygen generator 190 along with the humidifier means 200 may be housed within a portable bag pack 250. The oxygen generator 40 can be attached to the humidifier means 200 through the triggering means 90 when humidification of the generated oxygen is required. The housing of the oxygen generator 40 along with the humidifier means 200 is as shown in FIG. 6.

Various embodiments of the oxygen generator to be portable as it is composed of lighter material and is compact which makes the oxygen generator more reliable. Also, as the cartridges are small and compact, replacement of the cartridges are simple and quick. In addition, as the oxygen generator includes at least two cartridges, flow of oxygen is continuous, and every alternative cartridge can be easily replaced which keeps the flow of oxygen continuous.

Furthermore, as the oxygen generator does not include the storage tank, space of the oxygen generator is reduced and thereby the flow of generated oxygen is kept continuous. In addition, the oxygen generator includes the oxygen flow control knob which gives an option for the user to choose the level or rate of oxygen required which makes the oxygen cylinder more efficient.

Also, upon triggering the top seal, the pre-defined amount of water and the one or more chemicals gets combined automatically and reacts accordingly to generate the oxygen which saves time of the user to manually combine the one or more chemicals and the pre-defined amount of water for the generation of oxygen. Due to such non-intervention of human, accuracy on the ratio of the one or more chemicals and the pre-defined amount of water is maintained thereby making the oxygen generator more accurate.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependant on other acts may be performed in parallel with the other acts. The scope of embodiments is by no means limited by these specific examples.

We claim:

1. An oxygen generator 10 comprising:
a generator case 20 comprising at least two hollow chambers 30;
at least two cartridges 40, housed within the corresponding at least two hollow chambers 30, wherein each of the at least two cartridges 40 comprises:
a water chamber 50 configured to store a pre-defined amount of water;
a chemical chamber 60 mechanically coupled to the water chamber 50, and configured to store one or more chemicals in a pre-defined ratio,
wherein the water chamber 50 is placed above the chemical chamber 60 and is separated by a disc 70 and is pneumatically sealed;
a holding means 80 mechanically coupled to an interior top portion of the corresponding at least two cartridges 40;
a triggering means 90 mechanically held in place by the holding means 80, wherein the triggering means 90 comprises:
a top seal 100 configured to unlock a bottom seal 140 upon twisting a trigger connector 110 to a pre-defined angle;
a trigger valve 120 operatively coupled to the top seal 100, and configured to enable a process of generation of oxygen;
a hollow seal tube 130 located along a central axis of the corresponding at least two cartridges 40; and
the bottom seal 140 operatively coupled at a bottom of the hollow seal tube 130, and configured to allow the pre-defined amount of water to enter the chemical chamber 60 via a filter 150, wherein the filter 150 is fixed to the disc 70 within the chemical chamber 60,
wherein the hollow seal tube 130 is configured to:
allow flow of oxygen generated from chemical chamber 60 to the water chamber 50;
allow flow of oxygen generated in the chemical chamber 60 upon reaction of the pre-defined amount of water with the one or more chemicals to exit from the water chamber 50,
wherein the at least two cartridges 40 are placed in the corresponding at least two hollow chambers 30 of the generator case 20.

2. The oxygen generator 10 as claimed in claim 1, wherein the chemical chamber 60 of each of the at least two cartridges 40 further comprises a mechanical stirrer configured to keep the one or more chemicals in the chemical chamber 60 in constant mixing.

3. The oxygen generator 10 as claimed in claim 1, wherein the one or more chemicals comprises at least one of sodium percarbonate, potassium superoxide, peroxide species (hydrogen peroxide), urea-hydrogen peroxide and percarbamide peroxide.

4. The oxygen generator 10 as claimed in claim 1, wherein the one or more chemicals is on a form of one of a granular powder, pellets, powder in a pouch and candle stick.

5. The oxygen generator 10 as claimed in claim 1, wherein flow of oxygen in the hollow seal tube 130 is uni-directional and is controlled by the bottom seal 140.

6. The oxygen generator 10 as claimed in claim 1, wherein the trigger connector 110 is placed on a top surface of the generator case 20, and is configured to keep the connection of the at least two cartridges 40 in place during the generation of oxygen.

7. The oxygen generator 10 as claimed in claim 1, further comprising an oxygen flow control knob 160 operatively coupled to each of the at least two cartridges 40 via the corresponding trigger connector 110, and configured to control flow ratio of oxygen generated by each of the at least two cartridges 40.

8. The oxygen generator 10 as claimed in claim 7, further comprising a humidifier 180 operatively coupled to the oxygen flow control knob 160, and configured to impart humidity to the generated oxygen.

9. The oxygen generator 10 as claimed in claim 1, further comprising an oxygen level indicator 170 operatively coupled to each of the at least two cartridges 40, and configured to display oxygen level in each of the at least two cartridges 40.

10. The oxygen generator 10 as claimed in claim 1, further comprising an oxygen pressure regulator operatively coupled to each of the at least two cartridges 40, and configured to enable a uniform flow of oxygen.

11. The oxygen generator 10 as claimed in claim 1, further comprising a cooling mechanism operatively coupled to each of the at least two cartridges 40, and configured to dissipate heat from each of the at least two cartridges 40.

12. The oxygen generator 10 as claimed in claim 1, further comprising at least one storage tank operatively coupled to each of the at least two cartridges 40, and configured to enable a uniform flow of oxygen generated in the chemical chamber 60.

13. An oxygen generator 190 comprising:
at least one cartridge 40 comprising:
a water chamber 50 configured to store a pre-defined amount of water;
a chemical chamber 6 mechanically coupled to the water chamber 50, and configured to store one or more chemicals in a pre-defined ratio,
wherein the water chamber 50 is placed above the chemical chamber 60 and is separated by a disc 70 and is pneumatically sealed;
a holding means 80 mechanically coupled to an interior top portion of the corresponding at least one cartridge 40;
a triggering means 90 mechanically held in place by the holding means 80, wherein the triggering means 90 comprises:
a top seal 100 configured to unlock a bottom seal 140 upon twisting a trigger connector 110 to a pre-defined angle;
a trigger valve 120 operatively coupled to the top seal 100, and configured to enable a process of generation of oxygen;
a hollow seal tube 130 located along a central axis of the corresponding at least one cartridge 40; and
the bottom seal 140 operatively coupled at a bottom of the hollow seal tube 130, and configured to allow the pre-defined amount of water to enter the chemical chamber 60 via a filter 15, wherein the filter 150 is fixed to the disc 70 within the chemical chamber 60,
wherein the hollow seal tube 130 is configured to:
allow flow of oxygen generated from chemical chamber 60 to the water chamber 50;
allow flow of oxygen generated in the chemical chamber 60 upon reaction of the pre-defined amount of water with the one or more chemicals to exit from the water chamber;
a humidifier means 200 operatively coupled to the at least one cartridge 40 through the triggering means 90, and configured to impart humidity to the generated oxygen, wherein the humidifier means 200 comprises:
a container 210 configured to store water;
an oxygen flow control knob 160 operatively coupled to the container 210, and configured to control flow ratio of oxygen generated by the at least one cartridge 40;
an inlet valve 220 operatively coupled to a top surface 230 of the humidifier means 200, and configured to allow the flow of generated oxygen from the at least one cartridge 40 to the container 210; and
an outlet valve 240 operatively coupled to the top surface 230 of the humidifier means 200, and configured to allow the flow of humidified oxygen generated in the container 210.
